(19) United States
(12) Patent Application Publication
(10) Pub. No.: US 2023/0194532 A1
(43) Pub. Date: Jun. 22, 2023

(54) QUANTUM OPTICS PROFILES FOR SCREENING, DIAGNOSIS, AND PROGNOSIS OF DISEASES

(71) Applicants: King Abdullah University of Science and Technology, Thuwal (SA); King Saud University, Riyadh (SA)

(72) Inventors: Jean-Marc Nabholtz, Riyahdh (SA); Vladimir Bajic, Thuwal (SA); Roberto Incitti, Thuwal (SA); Khalid Alsaleh, Riyadh (SA)

(21) Appl. No.: 17/998,904

(22) PCT Filed: May 14, 2021
(86) PCT No.: PCT/IB2021/054158

§ 371 Date: Nov. 15, 2022

Related U.S. Application Data
(60) Provisional application No. 63/025,773, filed on May 15, 2020.

(51) Int. Cl. G01N 33/574; G01N 33/68; G16H 10/40

(52) U.S. Cl. CPC G01N 33/57415; G01N 33/6848; G16H 10/40; A61B 5/0075

(57) ABSTRACT

A method for diagnosing a disease, such as breast cancer, in a biological sample using spectroscopic data is described. The method involves computer-implemented method that runs an algorithm. The algorithm converts spectroscopic vibrational from the sample into a profile, and scores the profile using a pair of reference profiles. Based on the score and a threshold, it can be determined whether the subject from which the sample was obtained has a disease, and, if so, to what extent. The method also allows detection of early and pre-disease states in subjects based on the detection of signal of low concentration analytes that are indicative of early or incipient disease state. The method is non-invasive, non-subjective, and highly specific and sensitive. The method affords the application of a single standard of diagnostic accuracy, independent of the local availability of expert pathologists.

### CROSS REFERENCE TO RELATED APPLICATIONS

This application claims priority to and benefit of U.S. Provisional Application No. 63/025,773 filed on May 15, 2020, the disclosure of which is hereby incorporated herein by reference in its entirety.

### FIELD OF THE INVENTION

This invention is generally related to the screening and diagnosis of diseases, particularly assaying biological samples from a subject using quantum optics technology and scoring spectroscopic profiles (based on the molecular profile of the sample being analyzed) of the biological samples using batteries of algorithmic tests to determine whether the subject has a cancer disease, such as breast cancer, or is at risk of developing the cancer disease.

### BACKGROUND OF THE INVENTION

Over the last 55-65 years, there have been numerous efforts to improve upon the ability to assay biological samples to diagnose diseases in humans and other animals, so that effective treatment plans can be devised to arrest or eliminate diseases. Efforts have also been expended to develop the capacity to follow the evolution of a disease from the earliest time it is detectable in order to determine the most opportune time to intervene therapeutically and to choose for each patient the most efficient and effective form of therapy.

Despite all these efforts, methods of evaluating biological samples still exist that cannot monitor, in real-time, the effect of a treatment regimen on the evolution of a disease, such as cancer, from early stages, such as precancerous cells. “Real-time,” means that a system has the capacity to determine the rate of progression or regression of disease in each individual patient by comparing the properties of samples taken at different times from the patient. These methods cannot grade accurately the extent of progress of precancerous disease in an individual patient. The course of disease and the response of disease to therapy are only accessible via retrospective epidemiologic studies that give, at best, the average course of a disease and the average response of that disease to treatment.

Another problem with current diagnostic methods involves subjectivity, often manifested in the poor agreement between the conclusions of different pathologists examining the same data set. This subjectivity is particularly acute in diagnostic methods that involve microscopic viewing of samples. What complicates the diagnosis is that not all cells in a section of tissue or on a slide are affected equally, if affected at all. In addition, extensive changes in the chemical and physical attributes at the molecular level in cells may not appear as changes in the morphology of the cells. Further, and especially in the context of early diagnosis, the analyst is often looking for a few diseased cells amongst a large number of normal-appearing cells. Thus, a relationship exists between the validity of a diagnostic conclusion and the skill, diligence, and prior experience of the pathologist. There are no ways to control these variations so long as the fundamental method of diagnostic pathology remains a subjective process.

Lastly, many of the existing methods for obtaining samples involve invasive procedures that include, but are not limited to, endoscopies, biopsies, scrapings, and spinal taps.

Some attempted solutions involve immunohistochemistry. Immunohistochemistry involves using specific antibodies to detect expression and expression levels of known biomarkers in biological samples. However, immunohistochemistry has not sufficiently addressed the needs of the diagnostic industry owing to its requirements for accurate labeling, tagging, and specific knowledge of molecular biomarkers expressed in a disease, as well as the cumbersome production of antibodies that target those molecular biomarkers.

Other attempted solutions involve optical spectroscopic techniques to analyze biological samples. The diagnostic pathology services that are used to perform these analyses have inherent limitations. For example, the methods require knowledge of pre-existing associations between peaks, shapes of peaks, various wavelengths, and specific organs to produce meaningful diagnosis. Further complicating the analysis can be a requirement of the knowledge of different vibrational modes of biological molecules. Accordingly, these methods often require interpretation and/or analysis of spectrograms by expert pathologists, which in turn makes it difficult to provide high quality diagnostic pathology services in medically underserved regions of the world. As such, these services will not be available in the absence of trained pathologists in reasonable proximity to sites at which biological samples are collected. Therefore, when such services are performed without the assistance of trained pathologists, the quality of these services is extremely poor.

Another limitation of involves spectroscopic techniques with poor specificity and sensitivity. These spectroscopic techniques probe substructures present in molecules, not entire molecules. However, the occurrence of the same substructures in different molecules and rapid dephasing causes overlaps in the time and spectral responses, thereby limiting identification of specific molecules in complex samples. Other spectroscopic techniques are limited by their inability to reliably detect the presence of components that account for less than 5% by weight of the total mass of the sample.

Accordingly, there remains an unmet need to develop non-invasive, non-subjective, highly specific, and/or sensitive methods for the screening, diagnosis, and/or prognosis of diseases in humans or animals.

Therefore, it is an object of the present invention to provide improved diagnostic methods that overcome one or more of the problems discussed above.

It is also an object of the present invention to provide a non-invasive quantitative system and method analyzing biological samples and making interpretations about the presence or absence of disease, and optionally, if present, the stage (grade level) of the disease.

It is also an object of the present invention to provide a non-subjective, quantitative system and method analyzing biological samples and making interpretations about the presence or absence of disease, and optionally, if present, the stage (grade level) of the disease.

It is also an object of the present invention to provide a non-invasive, non-subjective, quantitative system and method analyzing biological samples and making interpretations about the presence or absence of disease, and optionally, if present, the stage (grade level) of the disease.

### SUMMARY OF THE INVENTION

A method for screening, diagnosis, and/or prognosis of a disease in a subject using molecular biomarkers in the subject's sample, is described. The subject can be a human or other animal, and the method can be performed in vitro or in vivo. The method combines an optical spectroscopic technique and a computer-implemented algorithm.

The optical spectroscopic technique assays the sample and generates vibrational frequencies that are indicative of the molecular profile of the sample. The optical spectroscopic technique is termed Quantum Optics High Spectrum Analysis (“QOHSA”). QOHSA is a technology type femto/atto-second infrared laser spectroscopy that can be applied to bio-fluids including blood (liquid biopsy). It is an elegant, non-invasive, and reproducible method allowing the capture of individualized molecular spectra with high throughput.

The computer-implemented algorithm takes the vibrational frequencies and generates a spectroscopic profile containing the vibrational frequencies. For a given sample, the computer-implemented algorithm assigns component scores for each vibrational frequency at each position in the profile, by comparing that vibrational frequency to that of its corresponding position in two reference spectroscopic profiles. A first reference spectroscopic profile and a second reference spectroscopic profile are generated using data obtained from a non-diseased sample and a diseased sample, respectively. The computer-implemented algorithm sums the component scores and compares the sum to a threshold. The subject is diagnosed with a disease if the sum is greater than the threshold. Otherwise, the subject is deemed disease-free.

The optical spectroscopic technique is carried out using, for example, a Raman analyzer with frequency span between 3100 cm⁻¹ and 900 cm⁻¹, such as 3050.855 cm⁻¹ and 929.527 cm⁻¹, inclusive, and 1101 points. These points govern the length of the profiles, which is determined by the range of the spectral frequencies and the spectral resolution of the instrument.

Also described are methods of using the methods described herein. The methods can be used for the screening, diagnosis, and/or prognosis of breast cancer.

### BRIEF DESCRIPTION OF THE DRAWINGS

FIGS. 1A and 1B are schematic diagrams of workflows useful in performing the diagnosis described herein. In FIG. 1A, sample is obtained and experimental spectroscopic technique performed in vitro. In FIG. 1B, spectroscopy technique is performed in vivo, i.e., in the body of the subject in situ.

### DETAILED DESCRIPTION OF THE INVENTION

### I. Definitions

“Non-subjective,” as used herein, and as relates to screening, diagnosis, and/or prognosis, means visual inspection of a sample and/or analysis of a spectrogram is not required to determine whether the sample is diseased or non-diseases.

### II. Method for Screening, Diagnosis, and/or Prognosis of Diseases

Described is a method for screening, diagnosis and/or prognosis of a disease in a subject using molecular biomarkers in the subject's sample. The subject can be a human or other animal. The method can be used on a sample in vitro or in vivo. Preferably, the method is non-invasive.

Currently, knowledge of each specific molecular biomarker is not required. The method can integrate all molecular biomarkers of profiles (which may be unique for a given subject at a given time) and correlate the results to a given question, which can be in a binary mode, such as existence or non-existence of a disease, such as cancer. An inquiry can also be along the lines of assessing the stage (grade level) of a disease, if a disease is detected. Accordingly, the method offers a significant improvement, because it provides a user with an elegant and streamlined quantitative analysis tool to probe a sample containing multiple molecules within a complex environment and to arrive at a diagnosis and/or prognosis without the need for (i) the user's knowledge of disease-specific molecular biomarkers in the sample and/or (ii) expertise in the interpretation of spectrograms. In some forms, knowledge of specific molecular biomarkers can be useful to link molecular profiles to specific biological modifications related to the asked binary question.

Preferably, the method involves an experimental analytical method, a computer-implemented method, or a combination thereof. In some forms, the method involves both an experimental analytical method and a computer-implemented method.

In some forms, the method involves (i) generating a spectroscopic profile of a subject's sample using an experimental analytical method, such that the spectroscopic profile contains one or more components, (ii) obtaining a general score of the spectroscopic profile using computer-implemented algorithm, and/or (iii) providing a diagnosis, prognosis, or both, of the disease based on the general score. Preferably, the components in the profiles can be ordered by wavenumber. The ordering can be by increasing or decreasing order.

In some forms, computing the general score involves using all the components of the spectroscopic profile. In some forms, computing the general score involves using some of the components that can be obtained within the spectroscopic profile by, for example, using progressively higher wavenumber resolutions for the same frequency range. This can also be achieved by using a larger frequency range and a higher wavenumber resolution.

In some forms, the method involves screening and diagnosis of breast cancer by performing a QOHSA measurement of a human sample, in particular those obtained in a non-invasive way, for example, using blood and computing a score based on the whole set of QOHSA “spectroscopic profiles at n variables” or from a part of it.

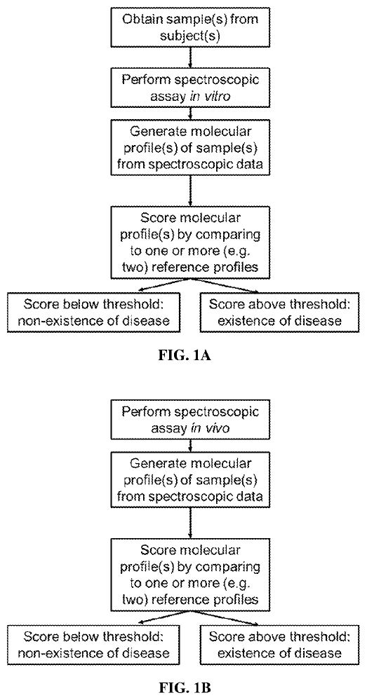

Referring FIGS. 1A and 1B, the experimental analytical method (such as spectroscopic assay) can be performed in vitro (FIG. 1A) or in vivo in the body of the subject (FIG. 1B). For a given sample, the method includes generating a spectroscopic profile containing data (such as vibrational frequencies) of the sample based on the spectroscopic assay, assigning a score for each profile by comparing, preferably, to two reference profiles containing data (such as vibrational frequencies), comparing the assigned score to a threshold, and/or determining whether the subject from which the sample was obtained has a disease, and optionally, if present, at what stage (grade level).

In some forms, an experimental analytical method (such as one described herein) involves a spectroscopic instrument, implement a spectroscopic technique, such as optical spectroscopy. In some forms, the spectroscopic instrument can be a Raman analyzer with frequency span between 3100 cm⁻¹ and 900 cm⁻¹, such as 3050.855 cm⁻¹ and 929.527 cm⁻¹, inclusive, and 1101 points.

In some forms, a computer-implemented algorithm (such as one described herein) can be used to general a spectroscopic profile based on the spectroscopic data obtained from a spectroscopic instrument. Preferably, the spectroscopic data can be a function of the molecular profile of the sample being analyzed.

The computer-implemented algorithm can also be used to generate one or more component scores by comparing the components of the spectroscopic profile of the sample with corresponding components in one or more reference spectroscopic profiles.

The following is a non-limiting example of how a reference spectroscopic profile is determined: for each of the features measured by the instrument, say the i-th feature, one identifies or computes the maximum and the minimum values observed over the control population, and denotes them by Max_i and Min_i, respectively. Then, for each sample, one computes a score composed of the number of features whose measured values are either higher than Max_i or lower than Min_i. This score is then used as a varying threshold to establish a receiver operating characteristic (ROC) curve from which the desired specificity can be chosen, obtaining the corresponding sensitivity. In another instance of the same method, controls whose scores are outliers can be removed.

Preferably, in some forms, the components of the spectroscopic profile of the sample and those in the one or more reference spectroscopic profiles contain vibrational frequencies. In some forms, the component scores can be summed to obtain a general score. In some forms, when the general score is greater than a threshold, the subject is deemed to have a disease, otherwise, the subject is disease-free. The general score is used to determine whether the subject has breast cancer.

In some forms, the computer-implemented algorithm generates component scores by comparing the components of the spectroscopic profile of the sample with corresponding components in two reference spectroscopic profiles, i.e., a first reference spectroscopic profile and a second reference spectroscopic profile. Preferably, the first reference spectroscopic sample contains upper bounds of spectroscopic data. Preferably, the second reference spectroscopic sample contains lower bounds of spectroscopic data. Preferably, in some forms, the components of the spectroscopic profile of the sample and those in the one or more reference spectroscopic profiles contain vibrational frequencies. In some forms, the component scores can be summed to obtain a general score. In some forms, when the general score is greater than a threshold, the subject is deemed to have a disease, otherwise, the subject is disease-free.

In some forms, at least one of the one or more reference spectroscopic profiles is generated using a non-diseased sample. In some forms, at least one of the one or more reference spectroscopic profiles is generated using a diseased sample. In some forms, at least one of the one or more reference spectroscopic profiles is generated using a cancerous sample. In some forms, the cancerous sample has a cancer selected from breast cancer, lung cancer, prostate cancer, colon cancer, skin cancer, blood cancer (such as leukemia and/or lymphoma), myeloma, and a combination thereof.

In some forms, at least one of the one or more reference profiles is from one or more individuals in the same population as the subject. In some forms, all the reference spectroscopic profiles are from one or more individuals in the same population as the subject. In some forms, at least one of the one or more reference spectroscopic profiles is from one or more individuals in a different population than the subject. In some forms, all the reference spectroscopic profiles are from one or more individuals in a different population than the subject.

The method can involve a probability in the screening, diagnosis, and/or prognosis, where a limited number of factors are used. For instance, for breast cancer, where a limited number of factors are used for classifications: clinical stage, hormonal receptors (estrogen and progesterone), amplification of the HER-2 gene and cell proliferation (mitotic index or Ki-67), this can lead to the definition of large subgroups, which are heterogeneous by nature, as breast cancer, on an individual patient basis, can be more complex than that. The first generation Quantum Optics technology, used here, allows to apprehend a partial molecular reality (the spectral results are totally reproducible for a given sampling) with a much wider array of biomarkers, some being specific to the disease or its biological consequences, some being specific of the host. Their computational integration, through batteries of algorithmic tests allows for the differentiation of individual profiles when asking relevant questions in a binary mode (more holistic approach).

i. Experimental Analytical Methods

The experimental analytical methods include one or more spectroscopic techniques. Examples of spectroscopic techniques include, but are not limited to, field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

In some preferred forms, the spectroscopic technique involves field-resolved spectroscopy (such as field-resolved infrared spectroscopy). In some forms, the spectroscopic technique involves frequency-resolved spectroscopy. In some forms, the spectroscopic technique involves infrared attenuated total reflectance. In some forms, the spectroscopic technique involves diffuse reflectance spectroscopy. In some forms, the spectroscopic technique involves multi-variable perturbation infrared techniques.

In some forms, the spectroscopic technique involves vibrational spectroscopy. In some forms, the vibrational spectroscopy includes infrared spectroscopy, such as near infrared spectroscopy, mid infrared, resonant frequency, and/or far infrared.

In some instances, spectroscopic methods probe the chemical substructures present in molecules, not entire molecules by detecting resonant vibrational responses to infrared or Raman excitation. However, the occurrence of the same fragments in different molecules and rapid dephasing causes overlaps in the time and spectral responses, thereby limiting identification of individual molecules in complex samples. These limitations can be overcome using field resolved spectroscopy (Pupeza, et al., Proc. Natl. Acad. Sci. USA 2020, 577, 52-59) that can detect distinct compounds in complex samples. Therefore, in preferred forms, the spectroscopic technique involves field-resolved spectroscopy (such as field-resolved infrared spectroscopy). In a preferred embodiment, the experimental analytical methods were performed as described in Pupeza, et al., Nature, 577: 52-59, 2020, the contents of which are herein incorporated by reference.

The spectroscopic instrument can be operated over a range of frequencies. The frequency ranges can span between about 14,000 cm⁻¹ and about 4000 cm⁻¹, between about 12,500 cm⁻¹ and about 4000 cm⁻¹, between about 4,000 cm⁻¹ and about 400 cm⁻¹, between about 4,000 cm⁻¹ and about 500 cm⁻¹, between about 4,000 cm⁻¹ and about 600 cm⁻¹, between about 4,000 cm⁻¹ and about 700 cm⁻¹, between about 4,000 cm⁻¹ and about 800 cm⁻¹, 4,000 cm⁻¹ and about 900 cm⁻¹, between 3,900 cm⁻¹ and about 500 cm⁻¹, between about 3,800 cm⁻¹ and about 600 cm⁻¹, between about 3,700 cm⁻¹ and about 700 cm⁻¹, between about 3,600 cm⁻¹ and about 800 cm⁻¹, 3,500 cm⁻¹ and about 900 cm⁻¹, 3,400 cm⁻¹ and about 900 cm⁻¹, between about 3,200 cm⁻¹ and about 900 cm⁻¹, between about 3,100 cm⁻¹ and about 900 cm⁻¹, between about 1,800 cm⁻¹ and about 750 cm⁻¹, between about 1,800 cm⁻¹ and about 800 cm⁼¹, between about 1700 cm⁻¹ and about 900 cm⁻¹. In some forms, the spectroscopic instrument can be a broadband femto-second resolved broadband infrared laser source, coupled with an infrared wave sampling system for ultra-sensitive molecular vibration spectroscopy. In some forms, the frequency scan ranges between about 3050.855 cm⁻¹ and about 929.527 cm⁻¹. In some forms, the spectroscopic instrument can be a Raman analyzer with frequency span between 3050.855 cm⁻¹ and 929.527 cm⁻¹, inclusive, and 1101 points.

Preferably, the spectroscopic instrument uses high resolution. High resolution can include detection levels at wavenumbers between 1 cm⁻¹ and 10 cm⁻¹, such as 1 cm⁻¹, 2 cm⁻¹, 3 cm⁻¹, 4 cm⁻¹, 5 cm⁻¹, 6 cm⁻¹, 7 cm⁻¹, 9 cm⁻¹, 9 cm⁻¹, or 10 cm⁻¹.

ii. Computer-Implemented Method

The computer-implemented method described herein, is not limited to any particular spectroscopic experimental analytical technique. The computer-implemented method implements an algorithm that is capable of general spectroscopic profiles using data generated from field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

The computer-implemented method can be performed on a computer that is capable of running the algorithm. The computer can be in physical proximity to the spectroscopic instrument that generates the data. The computer can also be at a remote location and is connected to the spectroscopic instrument via ethernet, bluetooth, near field communication, WiFi, integrated circuits, or a combination thereof.

Non-limiting examples of the processes performed by the algorithm are described in the Example. Briefly, the algorithm generates a spectroscopic profile containing one or more components, which is reflective of the molecular profile of the sample.

In the non-limiting example, the spectroscopic profile contains 1,101 features, determined from (begin_waveNumber (3050.856 cm⁻¹)−end_waveNumber (925.547 cm⁻¹))/(resolution (2 cm⁻¹)). The feature at each position in the spectroscopic profile corresponds to photon count intensity at that wavenumber. Accordingly, the length of the spectroscopic profile can be any value, but limited by the span of the frequency range and the resolution of the instrument.

In the non-limiting example described herein, the algorithm compares the spectroscopic profile of the sample being analyzed to two reference spectroscopic profiles. For each feature, at each position, it keeps track of a low component score (such as ComponentScore1) and a high component score (ComponentScore2). Upon traversing the length of the spectroscopic profile, the algorithm sums the low component score and the high component score. The algorithm then sums both component scores. If the sum is greater than a threshold, the subject is diagnosed with the existence of a disease. If the sum is less than the threshold, the subject is deemed disease-free.

### III. Methods of Using

The methods described herein can be used in the screening, diagnosis, and/or prognosis of a disease in a human or other animal. Suitable diseases include, but are not limited to, cancer, diabetes, atherosclerosis, Alzheimer's Disease, Parkinson's Disease, and chronic kidney disease. Exemplary cancers include, but are not limited to, a cancer selected from breast cancer, lung cancer, prostate cancer, colon cancer, skin cancer, blood cancer (such as leukemia and/or lymphoma), myeloma, and a combination thereof.

The sample to be analyzed can include the sample is selected from the group consisting of cells, blood, spittle/saliva, serum, plasma, urine, sputum, sweat, semen, synovial fluids, lymphatic fluids, cerebrospinal fluids, biopsy, stool, or combinations thereof.

In some forms, the subject is asymptomatic of a disease. In some forms, the subject presents one or more symptoms of a disease. Symptoms include, but are not limited to, breast pains, breast nodules, nipple discharge, weight loss, fatigue, anemia, or a combination thereof. In some forms, the subject has not had or has a prior history of having cancer.

In some forms, the subject is at risk (such as at high risk) of developing breast cancer. In some forms, the subject is exposed to one or more assays for identification of breast cancer.

A non-limiting example involves using particular patterns of QOHSA measurements for breast cancer screening and diagnosis. In some forms, the method involves using a combination of particular QOHSA measurement patterns of a variety of molecular biomarkers for breast cancer screening and diagnosis. These molecular biomarkers can be tested in tissue or in body fluids (such as blood, serum, plasma, urine, spittle, and sputum) and/or stool of patients with breast cancer. The format of one QOHSA measurement, termed spectroscopic profile, includes a vector of thousands of variables, each measuring the molecular profile of the bio-fluids at a given time.

Any appropriate method may be used to assess the target directly in the bio-specimen (because the sample preparation step can be skipped in some cases).

In some forms, the method is used as part of a regular checkup. Therefore, in some forms, the subject has not been diagnosed with a cancer (such as breast cancer) and, typically for those particular forms, it is not known that a subject has a hyperproliferative disorder, such as a breast neoplasm. In other forms, the individual is at risk for breast cancer, is suspected of having breast cancer, or has a personal or family history of cancer, including breast cancer, for example. In some forms, an individual is known to have cancer and the methods described herein are used to determine the type of BC, stage (grade level) of the breast cancer, treatment response to breast cancer, and/or prognosis. In some forms, the individual has already been diagnosed with breast cancer and may be subjected to surgery for breast cancer resection, and/or may undergo methods of the invention to survey the recurrence of breast cancer.

The present method has applicability for screening and/or diagnosing a wide range of diseases, and/or diseases at different stages. The method also allows detection of early and pre-disease states in subjects based on the detection of signal of low concentration analytes that are indicative of early or incipient disease state. For example, method can be used to detect the presence of abnormalities in samples that are below the level of detection by microscopic and optical spectroscopic examination of samples.

The methods can also be used to determine the stage (grade level) of a disease. Further, the computer-implemented methods can be applied to the results of a measure by any spectroscopic instrument, preferably using high-resolution spectroscopy. The spectroscopic instrument can be one that performs, among others, Fourier-transform infrared spectroscopy, Raman spectroscopy, or any device measuring either infrared intensities or Raman scattering coefficients against vibrational frequencies.

By virtue of the streamline process of the instant methods, which reduce and/or eliminate subjectivity, the requirement of expert analysis of spectrograms and/or samples under a microscope, the present methods make it possible to provide high quality screening and/or diagnostic pathology services in medically underserved regions of the world. The methods also provide a basis for immediate diagnostic decisions for patients and physicians, leading in turn to immediate implementation of next-step procedures and treatment. This means that patients and the examining clinician can know almost instantly whether or not the samples examined are diseased, non-diseased, and/or the stage (grade level) of disease, if present.

In some forms, the methods can be used to screen and/or diagnose a disease at a significantly high level of specificity and sensitivity. In some forms, this high level can be attributed to the expert medical advice involved in identifying the test data, the advanced experimental spectroscopic technique, and/or the expertise involved in the development and testing of the computer-implemented algorithm. In some forms field-resolved spectroscopy (such as field-resolved infrared spectroscopy) (Pupeza, et al., Proc. Natl. Acad. Sci. USA 2020, 577, 52-59) is used to assay samples at a significantly high level of sensitivity and specificity. This level can be much higher than in previously implemented spectroscopic and/or microscopic methods.

The present method can also be used to perform screenings and/or diagnosis in vivo. This is possible, because appropriate optical frequencies can be used to probe deeper tissue depths with optical non-invasive methods and the computer-implemented algorithm is well suited to analyze the output from the experiments. The medical importance of this aspect is not simply to allow for gathering immediate diagnostic information from a subject, but also to provide the ability to obtain more information from broader areas by examining samples inside the body than is available by taking biopsies or cells from the body and then examining them. For example, the act per se of biopsy of tissue distorts the remaining tissue and bleeding that accompanies a biopsy can distort a physician's view of the diseased tissue.

The disclosed methods can be further understood through the following numbered paragraphs.

1. A method for screening for and/or diagnosing a disease in a subject, the method comprising:

(i) generating a spectroscopic profile of the subject's sample or comparing a spectroscopic profile of the subject's sample with one or more reference spectroscopic profiles, wherein the spectroscopic profile, the one or more reference spectroscopic profiles, or both, comprise components,

(ii) obtaining a general score of the spectroscopic profile using a computer-implemented algorithm, and

(iii) providing a diagnosis, prognosis, or both, of the disease based on the general score.

2. The method of paragraph 1, wherein the diagnosis comprises comparing the general score to a threshold value, wherein the subject is diagnosed as having the disease when the general score is greater than the threshold.

3. The method of paragraphs 1 or 2, wherein obtaining the general score comprises using the computer-implemented algorithm to generate one or more component scores by comparing the components of the spectroscopic profile with corresponding components in at least one of the one or more reference spectroscopic profiles.

4. The method of paragraph 3, wherein the general score is obtained by summing the one or more component scores optionally using the computer-implemented algorithm, wherein when only one component score is available, the general score is that component score.

5. The method of any one of paragraphs 1 to 4, wherein the spectroscopic profile of the subject's sample, and the one or more reference profiles are generated using data from a spectroscopic technique that applies a frequency scan between about 14,000 cm⁻¹ and about 4000 cm⁻¹, between about 12,500 cm⁻¹ and about 4000 cm⁻¹, between about 4,000 cm⁻¹ and about 400 cm⁻¹, between about 4,000 cm⁻¹ and about 500 cm⁻¹, between about 4,000 cm⁻¹ and about 600 cm⁻¹, between about 4,000 cm⁻¹ and about 700 cm⁻¹, between about 4,000 cm⁻¹ and about 800 cm⁻¹, 4,000 cm⁻¹ and about 900 cm⁻¹, between 3,900 cm⁻¹ and about 500 cm⁻¹, between about 3,800 cm⁻¹ and about 600 cm⁻¹, between about 3,700 cm⁻¹ and about 700 cm⁻¹, between about 3,600 cm⁻¹ and about 800 cm⁻¹, 3,500 cm⁻¹ and about 900 cm⁻¹, 3,400 cm⁻¹ and about 900 cm⁻¹, between about 3,200 cm⁻¹ and about 900 cm⁻¹, between about 3,100 cm⁻¹ and about 900 cm⁻¹, between about 1,800 cm⁻¹ and about 750 cm⁻¹, between about 1,800 cm⁻¹ and about 800 cm⁻¹, between about 1700 cm⁻¹ and about 900 cm⁻¹.

6. The method of any one of paragraphs 1 to 5, wherein the spectroscopic profile of the subject's sample, and the one or more reference profiles are generated using data from a spectroscopic technique that applies a frequency scan between about 14,000 cm⁻¹ and about 4000 cm⁻¹, between about 12,500 cm⁻¹ and about 4000 cm⁻¹, between about 4,000 cm⁻¹ and about 400 cm⁻¹, or 3,100 cm⁻¹ and about 900 cm⁻¹.

7. The method of any one of paragraphs 1 to 6, wherein the spectroscopic profile of the subject's sample, and the one or more reference profiles are generated using data from a spectroscopic technique comprising field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

8. The method of paragraph 7, wherein the spectroscopic technique comprises vibrational spectroscopy.

9. The method of paragraph 8, wherein the vibrational spectroscopy comprises infrared spectroscopy, such as near infrared spectroscopy, mid infrared, resonant frequency, and/or far infrared.

10. The method of any one of paragraphs 1 to 9, wherein the components of the spectroscopic profile of the subject's sample contain vibrational frequencies.

11. The method of any one of paragraphs 1 to 10, wherein the components of at least one of the one or more reference spectroscopic profiles contain vibrational frequencies.

12. The method of any one of paragraphs 1 to 11, wherein at least one of the one or more reference spectroscopic profiles is generated using a non-diseased sample.

13. The method of any one of paragraphs 1 to 12, wherein at least one of the one or more reference spectroscopic profiles is generated using a diseased sample.

14. The method of any one of paragraphs 1 to 12, wherein at least one of the one or more reference spectroscopic profiles is generated using a cancerous sample.

15. The method of paragraph 14, wherein the cancerous sample has a cancer selected from the group consisting of breast cancer, lung cancer, prostate cancer, colon cancer, skin cancer, blood cancer (such as leukemia and/or lymphoma), myeloma, and a combination thereof.

16. The method of any one of paragraphs 1 to 15, wherein at least one of the one or more reference profiles is from one or more individuals in the same population as the subject.

17. The method of any one of paragraphs 1 to 14, wherein all the reference spectroscopic profiles are from one or more individuals in the same population as the subject.

18. The method of any one of paragraphs 1 to 13, wherein at least one of the one or more reference spectroscopic profiles is from one or more individuals in a different population than the subject.

19. The method of any one of paragraphs 1 to 15, or 18, wherein all the reference spectroscopic profiles are from one or more individuals in a different population than the subject.

20. The method of any one of paragraphs 1 to 19, wherein the general score is obtained by comparing the spectroscopic profile with a first reference spectroscopic profile and a second reference spectroscopic profile.

21. The method of paragraph 20, wherein the first reference spectroscopic profile contains upper bounds of spectroscopic data.

22. The method of paragraphs 20 or 21, wherein the second reference spectroscopic profile contains lower bounds of spectroscopic data.

23. The method of any one of paragraphs 1 to 22, wherein the subject is a human or other animal.

24. The method of any one of paragraphs 1 to 23, wherein the sample is in vitro or in vivo.

25. The method of any one of paragraphs 1 to 24, wherein the disease comprises cancer, diabetes, atherosclerosis, Alzheimer's Disease, Parkinson's Disease, or chronic kidney disease.

26. The method of any one of paragraphs 1 to 25, wherein the sample is selected from the group consisting of cells, blood, spittle/saliva, serum, plasma, urine, sputum, sweat, semen, synovial fluids, lymphatic fluids, cerebrospinal fluids, biopsy, stool, and combinations thereof.

27. The method of any one of paragraphs 1 to 26, wherein the subject is asymptomatic of the disease.

28. The method of any one of paragraphs 1 to 26, wherein the diagnosis is performed on the subject presenting symptoms of the disease.

29. The method of any one of paragraphs 1 to 28, wherein the subject has not had or has a prior history of having cancer.

30. The method of any one of paragraphs 1 to 27, or 29, wherein the subject exhibits one or more symptoms selected from the group consisting of breast pains, breast nodules, nipple discharge, weight loss, fatigue, anemia, or a combination thereof.

31. The method of any one of paragraphs 1 to 30, wherein the subject is at risk (such as at high risk) of developing breast cancer.

32. The method of any one of paragraphs 1 to 31, wherein the subject is exposed to one or more assays for identification of breast cancer.

33. The method of any one of paragraphs 1 to 32, wherein the general score is used to determine whether the subject has breast cancer.

34. The method of any one of paragraphs 1 to 33, wherein the components are ordered by wavenumbers.

Those skilled in the art will recognize, or be able to ascertain using no more than routine experimentation, many equivalents to the specific embodiments of the invention described herein. Such equivalents are intended to be encompassed by the following claims.

### EXAMPLES

The emergence of new infrared laser spectroscopic technology on liquid biopsy developed by the Max Planck Institute in Munich, Germany is opening new perspectives (Pupeza, et al., Nature, 577: 52-59, 2020). The “Broadband Infrared Diagnostics (BIRD)” technique makes it possible to use a femto-second molecular vibrational spectroscopy-type approach induced by controlled power lasers allowing to define “n variable biological profiles” acquired by an elegant non-invasive, sensitive, and low cost method in the context of cancer screening. This infrared (IR) and near infrared (NIR) laser spectroscopy technique relies on the scattering of photons as the incident light interacts with the target material. These interactions cause a frequency shift that reflects a particular molecular vibration energy. These vibrations are correlated with a specific molecular bond that makes it possible to build a “biochemical imprint”. Physiological or pathological changes lead to changes in the initial biochemistry and, consequently, changes in the spectra. These spectra are very detailed and integrated into a powerful computational approach allows to detect “n variable specific biological profiles” that can be potentially correlated with plasma differences related to the presence or absence of cancers or through the alteration of combined compositions which could serve as diagnostic markers.

The first-generation BIRD technology, a broadband femto-second infrared laser source, and an infrared wave sampling system for ultra-sensitive molecular vibration spectroscopy, and the design of a new Photon

Fluorescence Multi-Microscope System, are uniquely operational in Munich at the Max Planck Institute. Details are provided in Pupeza, et al., Nature, 577: 52-59, 2020).

This new performance regime of the technique is based on significant improvements in both the source and the detection of infrared radiation compared to the current state of this technique. The INFRALIGHT source presents a unique combination of high power/brightness, wide bandwidth and temporal coherence. High power/brightness can be an important feature for achieving high detection sensitivity and short acquisition time.

In a noise-reduced, noise-free spectroscopic apparatus, such as time-resolved field sampling based on a femto-second laser, the signal-to-noise ratio (S/N) is directly proportional to the coherent incident radiation power.

Therefore, high power means improved signal-to-noise ratio, which is very important for the detection of low concentration samples. Broad bandwidth is a prerequisite for recording almost complete files. In the case of complex organic mixtures consisting of hundreds of molecular species, this is crucial for the unequivocal identification of individual specimens.

These properties have been combined into a single source by the efficient non-linear conversion of ultra-short near-infrared pulses from a high-power femtosecond laser oscillator to the medium infrared range. Its first implementation covers the spectral range of 6.7 to 18 μm with an average power of 100 mW and holds the record for the highest power obtained with a coherent broadband source of this type. The power and bandwidth reported above can be significantly increased to improve the detection sensitivity and selectivity of a low concentration molecular sample.

The new source having dimensions of the order of the square meter will supplant considerably, in the two-way, advanced infrared technology synchrotron sources occupying areas of hundreds of square meters. The new source has significantly improved the acquisition sensitivity of infrared molecular fingerprints of cancer biomarkers in the blood.

### Example 1: Non-Invasive Diagnostic Test for Breast Cancer Detection Using Quantum Optics

The value of this approach in cancer screening was obtained in the context of breast cancer following the quantum optics analysis of a series of liquid biopsies from 67 healthy controls (absence of breast cancer by standard mammographic screening) and 28 patients with Stage I and II breast cancer (King Saud University, Riyadh, Saudi Arabia). Quantum Optics analysis was performed at the Max Planck Institute in Munich, Germany, and the definition of “Molecular Profiles at n variables” with the King Abdallah University for Science and Technology (KAUST) supercomputer in Jeddah, Saudi Arabia. A non-hierarchical data mining correlative analysis was performed to classify spectroscopic profile (that is based on the molecular profile of the sample being analyzed) between the two groups (controls versus patients) (n=1,101).

Several existing technologies, using femtosecond pulses of multi-octave infrared light and complete waveform measurements, allow for high-throughput spectroscopy measurements of biomolecules at low concentration, opening an avenue for cancer detection at early stage and therapy monitoring.

### Materials and Methods

In a collaboration between the Computational Bioscience Research Group at the King Abdullah University of Science and Technology and the Oncology Center at the King Saud University Medical City, a study was performed on a set of 56 breast cancer and 134 normal control samples.

Experimental Analysis

Experimental analysis of each sample was performed through quantum optics spectroscopy, using a version providing 1101 points of measure. Briefly, blood samples that were collected, were prepared, and were analyzed using quantum optics analysis according to the following steps: Samples were collected in tubes provided for this purpose up to 18.6 mL/sample per patient (tube EDTA K3). The samples were incubated at room temperature until the blood coagulated. The tubes were then centrifuged for 10 minutes at 7000 rpm at 4° C. and the supernatants were then aliquoted into micronic tubes (1 mL each serum) and stored at −80° C. No freeze-defreeze cycle was performed. Three aliquots of each serum sample at each sampling time (total 3 mL) were kept for the possibility of carrying out several independent experimental measurements. Tubes were then shipped to the Max Planck Institute (Munich, Germany) by batches, under cryopreservation with temperature controlled processes.

The experimental analysis were performed blindly at the Max Planck Institute. The blind was lifted at time of computational analysis.

Scoring Method

Algorithm 1 illustrates the procedure that was followed to score each sample.
The QOHSA measure of a sample is described by a vector f of 1101 components.
   (1) Normalize each sample vector components.
   (2) For each sample and its associate vector f to be analyzed, denote by f_i the i-th vector's component.
   (3) Two vectors a=(a_i) and b=(b_i) are provided.
   (4) Define 2 scores:

ComponentScore1/
S1 = sum(test_Low(f_i)), where
test_Low(f_i) = 1 if f_i < a_i
test_Low(f_1) = 0 otherwise
ComponentScore2/
S2 = sum(test_Hgh(f_i)), where
test_Hgh(f_i) = 1 if b_i < f_i
test_Hgh(f_1) = 0 otherwise

Prediction by Using Scores
Set a value T and assess that if S1+S2>T then the patient is predicted to have disease (such as breast cancer), or be disease-free (e.g. breast cancer-free) otherwise.

The components in the profiles were ordered by wavenumber. With regard to vectors a and b, the vectors' values were computed using the population data set (learning data set) that was used to perform the analysis described herein. The values in vectors a and b represent upper and lower bounds, respectively. The values are pertinent to the type of population that was used for the biological samples described herein. Coefficients and threshold were also computed using the same population data set.

It should be noted that the values in vectors a and b, the coefficients, and thresholds can be computed once and used for any population, or these values can be re-computed for applications targeted at other populations.

### RESULTS

The samples were assayed using quantum optics technology. By applying the scoring method described above, very high combined specificity and sensitivity values of recognition for disease/non-disease status were obtained. Those numbers depend on the underlying hypothesis on the population. In the absence of hypotheses, 92% specificity and 96% sensitivity can be considered very high, in terms of comparison with existing non-invasive tests. In the presence of strong outliers that cannot yet be considered true positives or true negatives, 96% specificity and 96% sensitivity were obtained, and considered very high.

An age-sensitive analysis found a sensitivity of 98% with a specificity of 97%.

Those skilled in the art will recognize, or be able to ascertain using no more than routine experimentation, many equivalents to the specific embodiments of the invention described herein. Such equivalents are intended to be encompassed by the following claims.

We claim:

1. A method for screening for and/or diagnosing a disease in a subject, the method comprising:
(i) generating a spectroscopic profile of the subject's sample or comparing a spectroscopic profile of the subject's sample with one or more reference spectroscopic profiles, wherein the spectroscopic profile, the one or more spectroscopic profiles, or both, comprise components,
(ii) obtaining a general score of the spectroscopic profile using a computer-implemented algorithm, and
(iii) providing a diagnosis, prognosis, or both, of the disease based on the general score.

2. The method of claim 1, wherein the diagnosis comprises comparing the general score to a threshold value, wherein the subject is diagnosed as having the disease when the general score is greater than the threshold.

3. The method of claim 1 or 2, wherein obtaining the general score comprises using the computer-implemented algorithm to generate one or more component scores by comparing the components of the spectroscopic profile with corresponding components in at least one of the one or more reference spectroscopic profiles.

4. The method of claim 3, wherein the general score is obtained by summing the one or more component scores optionally using the computer-implemented algorithm, wherein when only one component score is available, the general score is that component score.

5. The method of any one of claims 1 to 4, wherein the spectroscopic profile of the subject's sample, and the one or more reference profiles are generated using data from a spectroscopic technique that applies a frequency scan between about 14,000 cm⁻¹ and about 4000 cm⁻¹, between about 12,500 cm⁻¹ and about 4000 cm⁻¹, between about 4,000 cm⁻¹ and about 400 cm⁻¹, between about 4,000 cm⁻¹ and about 500 cm⁻¹, between about 4,000 cm⁻¹ and about 600 cm⁻¹, between about 4,000 cm⁻¹ and about 700 cm⁻¹, between about 4,000 cm⁻¹ and about 800 cm⁻¹, 4,000 cm⁻¹ and about 900 cm⁻¹, between 3,900 cm⁻¹ and about 500 cm⁻¹, between about 3,800 cm⁻¹ and about 600 cm⁻¹, between about 3,700 cm⁻¹ and about 700 cm⁻¹, between about 3,600 cm⁻¹ and about 800 cm⁻¹, 3,500 cm⁻¹ and about 900 cm⁻¹, 3,400 cm⁻¹ and about 900 cm⁻¹, between about 3,200 cm⁻¹ and about 900 cm⁻¹, between about 3,100 cm⁻¹ and about 900 cm⁻¹, between about 1,800 cm⁻¹ and about 750 cm⁻¹, between about 1,800 cm⁻¹ and about 800 cm⁻¹, between about 1700 cm⁻¹ and about 900 cm⁻¹.

6. The method of any one of claims 1 to 5, wherein the spectroscopic profile of the subject's sample, and the one or more reference profiles are generated using data from a spectroscopic technique that applies a frequency scan between about 14,000 cm⁻¹ and about 4000 cm⁻¹, between about 12,500 cm⁻¹ and about 4000 cm⁻¹, between about 4,000 cm⁻¹ and about 400 cm⁻¹, or 3,100 cm⁻¹ and about 900 cm⁻¹.

7. The method of any one of claims 1 to 6, wherein the spectroscopic profile of the subject's sample, and the one or more reference profiles are generated using data from a spectroscopic technique comprising field-resolved spectroscopy (such as field-resolved infrared spectroscopy), frequency-resolved spectroscopy, Fourier-transform infrared spectroscopy, Raman spectroscopy, infrared attenuated total reflectance, diffuse reflectance spectroscopy, and combinations thereof.

8. The method of claim 7, wherein the spectroscopic technique comprises vibrational spectroscopy.

9. The method of claim 8, wherein the vibrational spectroscopy comprises infrared spectroscopy, such as near infrared spectroscopy, mid infrared, resonant frequency, and/or far infrared.

10. The method of any one of claims 1 to 9, wherein the components of the spectroscopic profile of the subject's sample contain vibrational frequencies.

11. The method of any one of claims 1 to 10, wherein the components of at least one of the one or more reference spectroscopic profiles contain vibrational frequencies.

12. The method of any one of claims 1 to 11, wherein at least one of the one or more reference spectroscopic profiles is generated using a non-diseased sample.

13. The method of any one of claims 1 to 12, wherein at least one of the one or more reference spectroscopic profiles is generated using a diseased sample.

14. The method of any one of claims 1 to 12, wherein at least one of the one or more reference spectroscopic profiles is generated using a cancerous sample.

15. The method of claim 14, wherein the cancerous sample has a cancer selected from the group consisting of breast cancer, lung cancer, prostate cancer, colon cancer, skin cancer, blood cancer (such as leukemia and/or lymphoma), myeloma, and a combination thereof.

16. The method of any one of claims 1 to 15, wherein at least one of the one or more reference profiles is from one or more individuals in the same population as the subject.

17. The method of any one of claims 1 to 14, wherein all the reference spectroscopic profiles are from one or more individuals in the same population as the subject.

18. The method of any one of claims 1 to 13, wherein at least one of the one or more reference spectroscopic profiles is from one or more individuals in a different population than the subject.

19. The method of any one of claim 1 to 15, or 18, wherein all the reference spectroscopic profiles are from one or more individuals in a different population than the subject.

20. The method of any one of claims 1 to 19, wherein the general score is obtained by comparing the spectroscopic profile with a first reference spectroscopic profile and a second reference spectroscopic profile.

21. The method of claim 20, wherein the first reference spectroscopic profile contains upper bounds of spectroscopic data.

22. The method of claim 20 or 21, wherein the second reference spectroscopic profile contains lower bounds of spectroscopic data.

23. The method of any one of claims 1 to 22, wherein the subject is a human or other animal.

24. The method of any one of claims 1 to 23, wherein the sample is in vitro or in vivo.

25. The method of any one of claims 1 to 24, wherein the disease comprises cancer, diabetes, atherosclerosis, Alzheimer's Disease, Parkinson's Disease, or chronic kidney disease.

26. The method of any one of claims 1 to 25, wherein the sample is selected from the group consisting of cells, blood, spittle/saliva, serum, plasma, urine, sputum, sweat, semen, synovial fluids, lymphatic fluids, cerebrospinal fluids, biopsy, stool, and combinations thereof.

27. The method of any one of claims 1 to 26, wherein the subject is asymptomatic of the disease.

28. The method of any one of claims 1 to 26, wherein the diagnosis is performed on the subject presenting symptoms of the disease.

29. The method of any one of claims 1 to 28, wherein the subject has not had or has a prior history of having cancer.

30. The method of any one of claim 1 to 27, or 29, wherein the subject exhibits one or more symptoms selected from the group consisting of breast pains, breast nodules, nipple discharge, weight loss, fatigue, anemia, or a combination thereof.

31. The method of any one of claims 1 to 30, wherein the subject is at risk (such as at high risk) of developing breast cancer.

32. The method of any one of claims 1 to 31, wherein the subject is exposed to one or more assays for identification of breast cancer.

33. The method of any one of claims 1 to 32, wherein the general score is used to determine whether the subject has breast cancer.

34. The method of any one of claims 1 to 33, wherein the components are ordered by wavenumbers.
